(19) United States
(12) Patent Application Publication
(10) Pub. No.: US 2021/0052761 A1
(43) Pub. Date: Feb. 25, 2021

(54) INCENSE MATERIAL AND METHOD OF MANUFACTURE THEREOF

(71) Applicant: Richard Jellig, Baldwin, NY (US)

(72) Inventor: Richard Jellig, Baldwin, NY (US)

(21) Appl. No.: 16/998,655

(22) Filed: Aug. 20, 2020

Related U.S. Application Data
(60) Provisional application No. 62/890,262, filed on Aug. 22, 2019.

(51) Int. Cl. A61L 9/013; A61L 9/012

(52) U.S. Cl. CPC A61L 9/013; A61L 2101/32; A61L 9/012

(57) ABSTRACT

A method of manufacturing an incense material includes creating a mixture having five parts sawdust powder, one part adhesive, and at least six parts water. The method further includes baking the mixture and cooling the mixture to form the incense material.

### CROSS-REFERENCE TO RELATED APPLICATIONS

This application claims the benefit of and priority to U.S. Provisional Patent Application No. 62/890,262, filed on Aug. 22, 2019, the entire contents of which are incorporated by reference herein.

### BACKGROUND

### Technical Field

The disclosure is directed to an incense material and, more particularly, to a method of manufacture of the incense material.

### Background of Related Art

Burning incense material provides a pleasant aroma, thereby creating a particular atmosphere or masking unpleasant or undesirable odors. Typical prior art incense burners include a housing having a retaining element for securing an ignited piece of incense and means for catching falling incense ash. The housing may include a fan element for dispersing incense smoke or a cylindrical smokestack or chimney to surround a lit stick of incense. It would be advantageous to provide an incense material having structural rigidity and uniform shape that would reduce damage to the incense material and improve marketability thereof.

### SUMMARY

In accordance with an aspect of the present disclosure, a method of manufacturing an incense material includes creating a mixture having sawdust powder, in aspects five parts sawdust powder, an adhesive, in aspects one part adhesive, and water, in aspects at least six parts water. The method further includes baking the mixture and cooling the mixture to form the incense material.

In an aspect, baking the mixture may include baking the mixture for a suitable period of time and temperature, in aspects about 40 minutes at about 325° F.

In another aspect, the method may further include placing the mixture into an extruder.

In yet another aspect, placing the mixture into the extruder may include forming an elongate piece.

In still yet another aspect, forming the elongate piece may include forming a piece having a suitable length, in aspects about 21 inches.

In yet another aspect, the method may further include cutting the elongate piece into pieces, in aspects three inch pieces.

In an aspect, cooling the mixture may include cooling the mixture for a suitable period of time, in aspects about 24 hours.

In another aspect, creating the mixture may include creating a mixture having seven parts water.

In yet another aspect, creating the mixture may include creating a mixture having an adhesive, in aspects one part of an adhesive. In aspects, a natural binding agent may be used as the adhesive. Suitable natural binding agents include Machilus thunbergii powder, sandalwood powder, Tragacanth, Guar gum, Xanthum gum, or combinations thereof.

In still yet another aspect, creating the mixture may include creating a mixture including five parts sawdust processed from any suitable wood, in aspects a wood selected from apple, oak, maple, balsam, or any combinations thereof.

In accordance with another aspect of the present disclosure, an incense material is prepared by a process including creating a mixture having five parts sawdust powder, one part adhesive, and at least six parts water. The process further includes baking the mixture and cooling the mixture to form the incense material.

### BRIEF DESCRIPTION OF THE DRAWINGS

Various aspects and features of the present disclosure are described hereinbelow with reference to the drawings wherein like numerals designate identical or corresponding elements in each of the several views and:

FIG. 1 is a prior incense burner for use with an incense material in accordance with an aspect of the present disclosure;

FIG. 2 is the prior incense burner of FIG. 1 illustrating components thereof in phantom; and

FIG. 3 is a schematic flow chart of a method of making the incense material of FIG. 1.

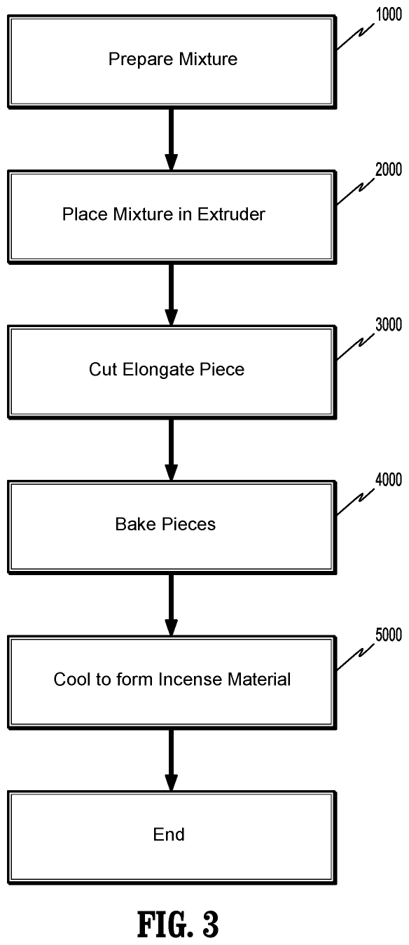

### DETAILED DESCRIPTION

The disclosed incense material will now be described in detail with reference to the drawings in which like reference numerals designate identical or corresponding elements in each of the several views. However, it is to be understood that the disclosed aspects are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure. In addition, directional terms such as front, rear, upper, lower, top, bottom, distal, proximal, and similar terms are used to assist in understanding the description and are not intended to limit the disclosure.

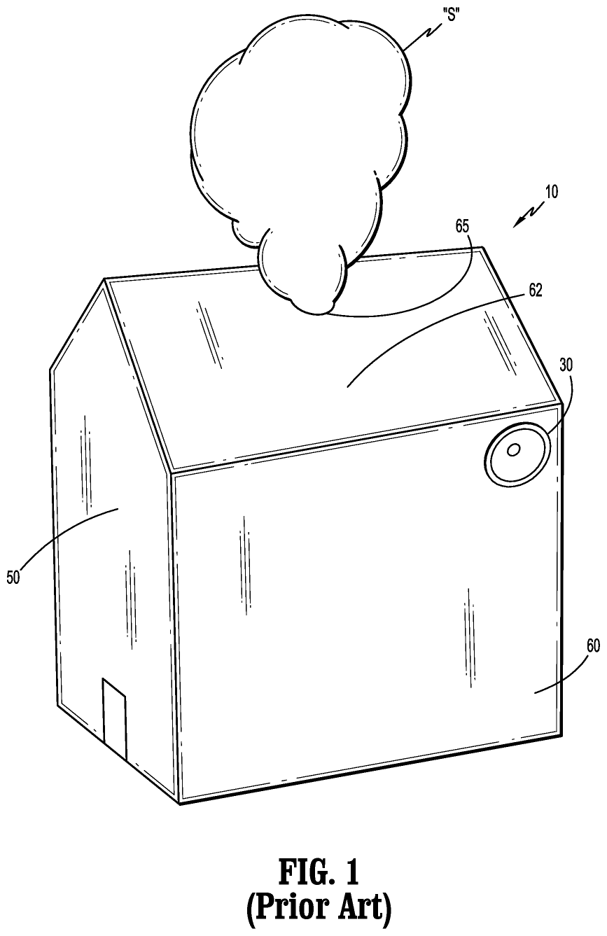

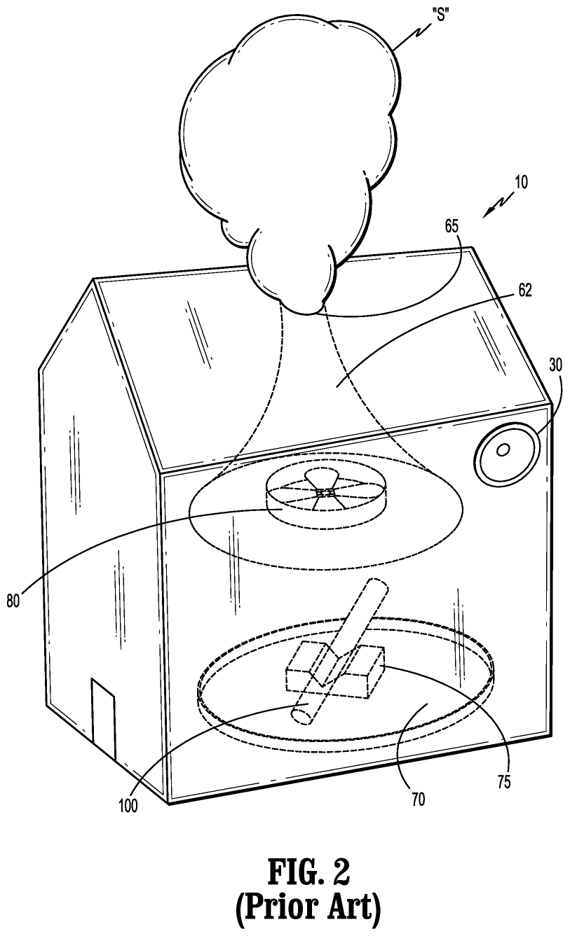

With reference to FIGS. 1 and 2, a prior art incense burner 10 may be utilized for use with an incense material 100 (shown in phantom) in accordance with an aspect of the present disclosure. The incense burner 10 generally includes a housing 50 defining a chamber 60 therein. The chamber 60 is configured to receive the incense material 100 therein. The housing 50 may be formed of any material such as, e.g., aluminum or titanium, suitable for withstanding heat and residual incense ash and easy cleaning. The chamber 60 is in communication with an aperture 65 configured to direct the smoke “S” out of the chamber 60. The chamber 60 may further include a passage 62 (shown in phantom) that may be tapered to direct the smoke “S” of the incense material towards the aperture 65. The aperture 65 may be dimensioned large enough to allow air to flow freely through the aperture 65, while small enough to reduce or minimize the amount of incense ash that can flow therethrough.

With particular reference to FIG. 2, the chamber 60 may include a tray 70 (shown in phantom) having a support member 75 (shown in phantom) configured to support the incense material 100 thereon. The tray 70 is configured to collect any residual incense ash. The incense burner 10 may include a door panel (not shown) providing access to the chamber 60 in order to place or remove the incense material to and from the chamber 60.

In aspects, the housing 50 may be further provided with a blower 80 (shown in phantom) in the form of an electrically driven fan that is mounted in a suitable manner inside of the housing 50 and connected to a suitable battery. The blower 80 is oriented such that it directs the flow of air towards the aperture 65. As a result of the upward flow of air, incense smoke “S” generated by the burning incense material 100 (positioned in holders 50) is drawn towards the aperture 65. In an aspect, the housing 50 may further include a sound system unit 30 external to the chamber 60. The sound system unit 30 may be configured to provide a mixture of sounds such as, e.g., sound of burning fireplace logs.

The incense material 100 in accordance with an aspect of the present disclosure is formed of a mixture including sawdust in a finely ground powder form, adhesive, and distilled water (step 1000). In aspects, the sawdust may be prepared by processing wood chunks of any suitable wood, e.g., apple, oak, maple, balsam, or any combination thereof, into chips, which are then fed into a hammer mill, a grinding mill, or any combination thereof. The final sawdust is obtained by passing the powder through a sieve with a nominal mesh aperture from about 200 microns to about 350 microns, in aspects from about 180 microns to about 330 microns, in other aspects from about 225 microns to about 300 microns.

The adhesive for use in the incense material may be any suitable adhesive within the purview of the skilled artisan. As used herein, an adhesive may sometimes also be referred to as a “binding agent.” In aspects, a suitable adhesive includes a natural binding agent such as, e.g., Machilus thunbergii powder. For example, a bark of Machilus thunbergii in a finely ground powder form may serve as a binding agent. Alternatively, sandalwood powder may also be used as a binding agent. Other suitable binding agents which may be used as an adhesive in the mixture include, for example, Tragacanth, Guar gum, Xanthum gum, or combinations thereof.

The mixture may contain suitable amounts of sawdust powder, adhesive, and water, in aspects distilled water. For example, in some aspects the mixture may contain 5 parts sawdust powder, 1 part adhesive, and 6 to 7 parts distilled water. In some aspects, the mixture may contain 2 to 8 parts sawdust powder, in some cases 4 to 6 parts sawdust powder; 0.5 to 3 parts adhesive, in some cases 0.75 part to 2 parts adhesive, in some cases 1 part adhesive; and 4 to 10 parts water, in aspects 5 to 8 parts water, in some cases 6 to 7 parts water.

Methods for forming the mixture are within the purview of those skilled in the art and include, but are not limited to, mixing, blending, sonication, heating, combinations thereof, and the like.

Once formed, the mixture is placed in an extruder which forms elongate pieces having a suitable length. The length may be any length from about 12 inches to about 36 inches, in aspects from about 16 inches to about 32 inches, in other aspects from about 18 inches to about 26 inches, with about 21 inches being extruded in some aspects (step 2000). Each piece may then be cut into smaller pieces having a length of, e.g., from about 2 inches to about 8 inches, in aspects from about 2.5 inches to about 5 inches, in some aspects about 3 inches (step 3000). Thereafter, the pieces are baked for a suitable period of time. For example the pieces may be baked at a temperature from about 275° F. to about 375° F., in aspects from about 300° F. to about 350° F., in some aspects about 325° F., for a period of time from about 20 minutes to about 60 minutes, in aspects from about 30 minutes to about 50 minutes, in some aspects for about 40 minutes (step 4000). In some aspects, the pieces may be baked at a temperature of about 325° F. for a period of about 40 minutes.

The pieces are then cooled at, e.g., room temperature, for a suitable period of time, for example from about 12 hours to about 36 hours, in aspects from about 18 hours to about 30 hours, in some aspects about 24 hours, prior to use (step 5000). The incense material 100 formed under such a process provides structural rigidity that reduces damage to the incense material 100 during transport to any retail merchants and/or the final consumer. In addition, the incense material 100 provides uniform shape and surface roughness, which improves marketability of the incense material 100.

Persons skilled in the art will understand that the incense material and methods specifically described herein and illustrated in the accompanying drawings are non-limiting exemplary aspects. It is envisioned that the elements and features illustrated or described in connection with one exemplary aspect may be combined with the elements and features of another without departing from the scope of the disclosure. For example, while the incense material 100 is formed as an elongate shape, it will be appreciated, however, that the incense material 100 may be made in various shapes and profiles including without limitation rectangular, square, cubic, rectangular prisms, cylinders, cones, pyramidal, even spherical and many variations thereof without departing from the disclosure.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as examples of particular aspects. Those skilled in the art will envision other modifications within the scope and spirit of the claims appended hereto.

What is claimed is:

1. An incense material as disclosed herein.

2. A method of manufacturing an incense material as disclosed herein.

3. A method of manufacturing an incense material comprising:
creating a mixture having:
five parts sawdust powder;
one part adhesive; and
at least six parts water;
baking the mixture; and
cooling the mixture to form the incense material.

4. The method according to claim 3, wherein baking the mixture includes baking the mixture for about 40 minutes at about 325° F.

5. The method according to claim 3, further comprising placing the mixture into an extruder.

6. The method according to claim 5, wherein placing the mixture into the extruder includes forming an elongate piece.

7. The method according to claim 6, wherein forming the elongate piece includes forming a piece having a length of about 21 inches.

8. The method according to claim 7, further comprising cutting the elongate piece into smaller pieces.

9. The method according to claim 3, wherein the cooling the mixture includes cooling the mixture for about 24 hours.

10. The method according to claim 3, wherein creating the mixture includes creating a mixture including seven parts water.

11. The method according to claim 3, wherein creating the mixture includes creating a mixture having one part adhesive, the adhesive including a binding agent selected from Machilus thunbergii powder, sandalwood powder, Tragacanth, Guar gum, Xanthum gum, or combinations thereof.

12. The method according to claim 3, wherein creating the mixture includes creating a sawdust processed from wood selected from apple, oak, maple, balsam, or any combination thereof.

13. An incense material prepared by a process comprising:
creating a mixture having:
five parts sawdust powder;
one part adhesive; and
at least six parts water;
baking the mixture; and
cooling the mixture to form the incense material.
